(19) United States
(12) Patent Application Publication
(10) Pub. No.: US 2008/0135808 A1
(43) Pub. Date: Jun. 12, 2008

(54) Immersion oil, method of producing immersion oil, method of reserving immersion oil and reserving vessel for immersion oil

(75) Inventors: Hiroaki Kinoshita, Tokyo-to (JP); Atsushi Niwa, Kyoto-shi (JP); Masahiro Satoh, Ohtsu-shi (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: Olympus Corporation, Tokyo (JP)

(21) Appl. No.: 11/986,910

(22) Filed: Nov. 27, 2007

(30) Foreign Application Priority Data
Nov. 29, 2006 (JP) 2006-321473

(51) Int. Cl. G01N 33/00; B65D 85/00

(52) U.S. Cl. 252/408.1; 206/524.4

(57) ABSTRACT

The immersion oil has refractive index which is 1.70 or more, and is low autofluorescence. Preferably, the main ingredient is iodine compounds (diiodomethane), and a solid having a high refractive index such as sulfur and the like is dissolved or dispersed into the immersion oil. The production method of the immersion oil has a purification process, in which impurities are removed preferably by adsorbent, distillation or recrystallized. In the method of preservation of the immersion oil, it is always contacted with the adsorbent. The preservation container of the immersion oil contains an adsorbent which is always contacted with the immersion oil.

This application claims benefits of Japanese Patent Application No. 2006-321473 filed in Japan on Nov. 29, 2006, the contents of which are hereby incorporated by reference.

### BACKGROUND OF THE INVENTION

1. Field of the Invention

The present invention is related to an immersion oil having a high refractive index and a low fluorescence which has not been available so far, and is used for an observation or a measurement method by various kinds of optical instruments, such optical system as a microscope, a fluorescence microscope, a microscope, etc., a production method of the immersion oil, a method of preservation of the immersion oil, and a preservation vessel of the immersion oil.

2. Description of the Related Art

Various kinds of immersion oil have been used for optical instruments such as microscope and the like from so far. Immersion oil is a substance that is used in order to improve optical characteristics by filling up a space where an air exists between optical components, or between an optical component and a specimen etc. in an optical system. For example, in case using an optical device for observing a specimen close to a lens such as a microscope, a refractive index of the immersion oil which exists between an objective lens and a specimen is influenced. Accordingly, in order raise its resolving power, an oil having a high refractive index of the immersion oil existing between the objective lens and the specimen has been used.

When an immersion oil is used, compared with the case that the immersion oil is not used, not only it can make an optical aberration small substantially, but also it can raise a magnification of a microscope by increasing numerical aperture of the objective lens.

Recently, in the field of a microscope, a fluorescence observation in which fluorescence emitted from a specimen and an object is observed by using excitation light generated by illuminating light having a specific wavelength has been used widely. In the field in which an observation, analysis, and measurement of biological materials (a living body cell, DNA, RNA etc.) are carried out by the fluorescence observation, and a detection and analysis of very small amount of a substance are carried out in a semiconductor manufacturing process, by a particle fluorescent measurement and multiple coloring of a fluorescence pigment using various kinds of fluorescence pigments, a simultaneous analysis of a living body function etc. has been made. Thus, importance about the technology by which weak fluorescence can be observed or measured correctly by using a wide wavelength band increases. Particularly, in an observation called as one particle fluorescence observation that is very weak fluorescence observation, autofluorescence of an immersion oil and the like which may cause a noise becomes large relatively as a background noise of an observation image since the fluorescence from a specimen is very weak. Thus, there is a problem that an observation cannot be made substantially.

An apparatus equipped with function for observing, imaging a sample such as a microscope, and an apparatus that measures a sample, and counts a measurement result, and an optical device which various kinds of functions were added

have been developed. In these optical apparatus, an exact measurement method which is not influenced by a noise at the time of measurement is required.

In fluorescence observation or fluorescence measurement, a bright optical system having a large number of numerical aperture which can measure weak fluorescence correctly has been required. For example, an optical system having numerical aperture NA=1.3 or more, and preferably NA=1.6 or more is demanded. As an optical system having such numerical aperture, immersion oil having high refractive index such as 1.7 or more, preferably 1.75 to about 1.8, is necessary.

As an observing method which requires the immersion oil having such high refractive index and low autofluorescence, for example, an application shown in Publication of the Japanese unexamined patent application, Toku Kai. 2005-189237 is effective. In such application, it is required that in an optical path between a glass constituting an optical system and a specimen, an immersion oil having a refractive index equivalent to the refractive index the glass is filled.

Here, the immersion oil in which an refractive index exceeds 1.8 can not be realized unless a substance having toxicity such as an arsenic compound and the like is added. Accordingly, it is considered that a range of the refractive index of the immersion oil having a high refractive index which can be practically used is about 1.7 to 1.8.

As the immersion oil used conventionally for a microscope observation, what consists of liquefied polyolefin and aromatic compounds has been known. Such material has been shown for example in Japanese published unexamined patent application Toku Kai Hei 11-160623, Japanese published unexamined patent application Toku Kai Hei 11-218685, Japanese published unexamined patent application Toku Kai Hei 11-269317, etc.

As an immersion oil having an refractive index of 1.75 around, a material containing diode methane and sulfur etc., has been proposed, and generally widely used. For example, Series M made by Cargille Co. and the like have been known.

### SUMMARY OF THE INVENTION

The immersion oil according to the present invention is characterized in that the immersion oil has a refractive index which is 1.70 or more, and low autofluorescence.

The immersion oil according to the present invention is characterized in that it has a refractive index which is 1.78 or more, and low autofluorescence.

In the immersion oil of the present invention, it is desired that an iodine compound is a main ingredient.

In the immersion oil of the present invention, it is desired that the iodine compound is diiodomethane.

In the immersion oil of the present invention, it is desired that a solid having a high refractive index is dissolved or dispersed.

In the immersion oil of the present invention, it is desired that the solid having a high refractive index is sulfur.

The production method of the immersion oil according to the present invention is characterized in that in one of the production method of the immersion oil of the present inventions, it has a purification process.

In the immersion oil of the present invention, it is desired that the purification process is a process for removing impurities by adsorbent.

In the immersion oil of the present invention, it is desired that the purification process is a process for removing impurities by distillation.

In the immersion oil of the present invention, it is desired that the purification process is a process for removing impurities by recrystallization.

The method of preservation of the immersion oil according to the present invention is characterized in that an adsorbent is always contacted with the immersion oil in the method of preservation of one of the immersion oil of the present inventions mentioned above, or the immersion oil manufactured by one of the production methods of the present inventions mentioned above.

The method of preservation of the immersion oil according to the present invention is characterized in that the immersion oil is made contacted with an absorbent whenever the immersion oil is taken out from the preservation container to outside, in the method of preservation of one of the immersion oil of the present inventions mentioned above, or the immersion oil manufactured by one of the production methods of the present inventions mentioned above.

The preservation vessel of the immersion oil is characterized in that the preservation vessel contains an adsorbent which is always contacted with the immersion oil in the vessel for the immersion oil manufactured by one of the production methods of the present inventions mentioned above.

The preservation vessel of the immersion oil is characterized in that the preservation vessel contains an adsorbent which is contacted with the immersion oil whenever it is taken out from the immersion oil in the preservation vessel.

In the preservation vessel of the immersion oil according to the present invention, it is desired that a portion which contains an adsorbent contacted with the immersion oil whenever it is taken out can be attached and detached to the main body of the preservation vessel.

According to the present invention, an immersion oil that has a high refractive index and low autofluorescence, (that is, generation of light quantity of autofluorescence is small), and satisfies for other conditions required for the immersion oil for optical instruments, and is useful as an immersion oil for various kinds of optical systems including an immersion oil for microscopes, and the production method of the immersion oil can be obtained. Furthermore, the method of preservation of the immersion oil and its preservation vessel by which observation with small background noise by autofluorescence can be carried out for a long period, can be obtained.

These and other features and advantages of the present invention will become apparent from the following description of the preferred embodiments when taken in conjunction with the accompanying drawings.

### BRIEF DESCRIPTION OF THE DRAWINGS

FIG. 1 is a conceptual diagram showing an outlined composition of a preservation vessel of an immersion oil concerning one of embodiments according to the present invention.

FIG. 2 is a conceptual diagram showing an outlined composition of a preservation vessel of an immersion oil concerning another embodiment according to the present invention.

### DETAILED DESCRIPTION OF THE PREFERRED EMBODIMENTS

Hereafter, the present invention will be explained in detail by embodiments of the invention. The immersion oil according to the present invention is characterized in that it has a refractive index which is 1.70 or more, and preferably 1.78 or more, and low autofluorescence. In more detailed explanation, it is an immersion oil that contains an iodine compound as a main ingredient, wherein a solid having a high refractive index is dissolved or distributed. As an iodine compound, it is desired that it is diiodomethane.

Here, in the present invention “low autofluorescence” is defined as follows.

In the very weak fluorescence observation apparatus mentioned above, a signal will be simply hidden in weak noises since the signal is very weak. The signal or the noise as a value of amount of the fluorescence varies with time, respectively. Here, “fluctuation” is defined such that it is in proportion to the square root of the variation of the quantity of light. When the absolute value of amount of the fluorescence from an object of observation/measurement of a specimen is large, a rate of “fluctuation” to the signal or the noise is relatively low. Generally, when a signal or a noise is very weak, the rate of “fluctuation” by the noise to the signal will become high.

When the absolute value of amount of the fluorescence from the object of observation/measurement of a specimen is small, the absolute value of the noise and “fluctuation”, and the unnecessary absolute value of the signal and “fluctuation” bring a problem.

In fluorescence microscope observation, most of the noises are occupied by sum total of a noise other than the object of observation of a specimen, a noise of autofluorescence of an objective lens and an optical element, a noise of the autofluorescence of a cover glass, a noise of the objective lens using an immersion oil, and a noise of the autofluorescence of the immersion oil when using what is called an oil objective.

In the present invention, “low autofluorescence” is defined such that there is little quantity of light of fluorescence other than the fluorescence from the object of observation/measurement of a specimen. Furthermore, in the present invention, it is assumed that there is a noise by the autofluorescence of immersion oil among fluorescence other than the fluorescence for observation/measurement of the specimen.

Here, a value of a signal of an object to be observed, and a value of a noise of the autofluorescence noise in the background noise containing the noise originated from the immersion oil are adopted for defining S/N ratio, and it is defined as follows.

(S−s)/(B+b) (1)

Here, S is an average quantity of light of a signal;

### B is an average amount of light of the background noise containing the noise originated from the immersion oil;

s a fluctuation of an average quantity of light of a signal;
and b is a fluctuation of an average amount of light of the background noise containing the noise originated from the immersion oil. That is to say, the signal is set as S±s, the noise is set as B±b, and a ratio of the lower limit of a signal value to the upper limit of a noise value is defined as an S/N ratio.

As a result of study, it has been concluded that in a single particle observation by vertical-illumination fluorescence etc., that is, in an application having a very low S/N ratio, namely, the formula, (1) mentioned above, a level of “low autofluorescence” which is required to the application wherein (S−s)/(B+b)≦2 is satisfied is as follows;

B′/B≦0.3

Here, Bib and B′±b′ is a background noise containing the background noise containing a noise originated from an immersion oil (Series M immersion oil made by Cargille Co.) that has been generally used now, and the noise of the immersion oil originated from the present invention. Thus, the noise level is expressed in such way, because an absolute value might be changed according to a measuring instrument, if it is expressed in terms of the absolute value of a noise level.

Furthermore, it has been traced and concluded that in applications such as a fluorescence observation/measurement by a low dye-concentration specimen as a time lapse and the like, or by weak excitation light which will be a trend from now on, namely, in the formula (I) mentioned above, a level of “low autofluorescence” which is required to the application, wherein (S−s)/(B+b)≦2 is satisfied is as follows;

B′/B≦0.5

Furthermore, it has been traced and concluded that in a usual application in fluorescence observation, namely, in the formula (I) mentioned above, a level of “low autofluorescence” which is required to the application wherein (S−s)/(B+b)≦5 is satisfied is as follows;

B′/B≦0.7

### Namely, it has been confirmed that it is required that B′/B is less than 0.3, 0.5, and 0.7 in noise level, respectively, and that this noise level has been attained for the first time by the immersion oil according to the present invention,

in each of cases of applications wherein (S−s)/(B+b)≦2, or 3, or 5 is satisfied, wherein B represents an average value of the fluorescence quantity of light in case that an immersion oil that is produced by Cargille Co. before the-present-invention is used, and a specimen does not exist;

B′ represents an average of the fluorescence quantity of light in case that an immersion oil according to the present invention is used, and a specimen does not exist.

This immersion oil can be obtained with a production method having a purification process. The purification process can be worked independently to the main ingredients and a solid having a high refractive index, respectively, or, it can also be worked after the solid having a high refractive index is dissolved or dispersed in the main ingredient. As a purification process, for example, an impurities removing method by adsorbent, an impurities removing method by distillation, an impurities removing method by recrystallization, an impurities removing method by sublimation, and the like can be used. Two or more kinds of these impurities removing methods can be also combined.

In the case of the impurities removing by adsorbent, for example, as adsorbent, silica gel, activated carbon, alumina, clay, and the like can be used. Silica gel and activated carbon are desirable.

As types of usage of adsorbent, the following types and the like can be used; for example, the adsorbent of cylindrical form arranged and a substance containing impurities is passed through its inside; the adsorbent is suspended in the substance having contained impurities, and separately it is filtered by using a filtration means; or the adsorbent is put into a vessel constituted so that a liquid may pass freely according to difference of a grain size etc., and the adsorbent may not leak, and it is immersed in a substance having contained impurities,

As for an amount of the adsorbent used, 0.01 g˜5 g are desirable, and 0.05 g˜3 g are more desirable to 10 ml of substances containing impurities. When it is less than 0.01 g, removal effect of impurities is small, and if it is more than 5 g, recovery decreases, or, a dissolved substance may deposit. In case that impurities are removed by distillation, the distillation can be carried out under normal pressure or reduced pressure. In order to suppress decomposition under distillation, it is desired that it is carried out under atmosphere of inactive gas represented by nitrogen or argon. In case of impurities removing method by recrystallization, any solvent can be used if there is no reactivity with material containing impurity. It is desirable to use especially toluene or ethyl acetate. As an embodiment for refining the immersion oil according to the present invention, it is desired that sulfur from which impurities were removed by recrystallization is dissolved in diiodomethane from which impurities were removed by adsorbent or distillation.

### By such way as mentioned above, control of refractive index becomes easy, and deposit of sulfur can be prevented.

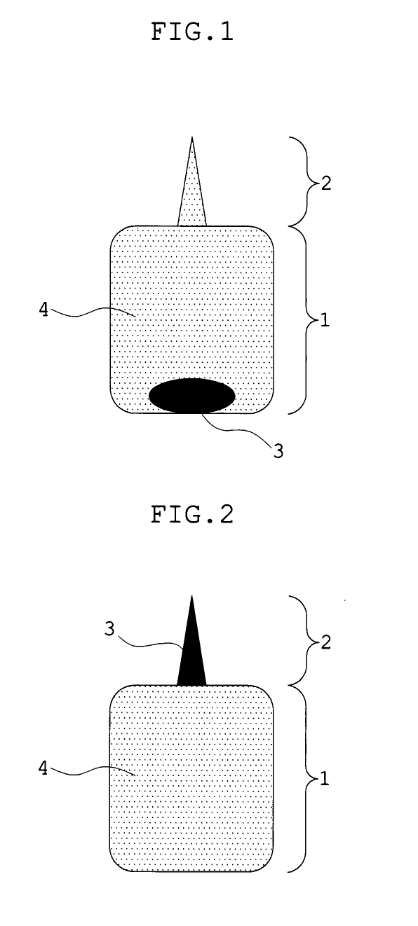

The method of preservation according to the present invention is how to preserve the immersion oil of the present invention mentioned above. It is characterized in that an adsorbent is always contacted with the immersion oil. The vessel for preservation of the present invention is a vessel for preserving the immersion oil of the present invention mentioned above. It is characterized in that it contains an adsorbent which is always contacted with the immersion oil. A an adsorbent, as mentioned above, silica gel, activated carbon, alumina, clay and the like can be used, It is desired that silica gel or activated carbon is used. As to a form for containing an adsorbent, it is not particularly limited, if it is a form that adsorbent is not mixed when using immersion oil. However, as shown in FIG. 1, for example, it is desired that it is contained in a meshed bag of non-textile that the immersion oil passes freely but adsorbent 3 does not leak and, it is arranged in a main body 1 of a vessel for preservation. In FIG. 1, numeral 2 represents a nozzle part.

Another method of preservation according to the present invention is a method for preserving the immersion oil of the present invention mentioned above, it is characterized in that it is made contacted with an absorbent whenever the immersion oil is taken out from the preservation vessel to outside. Another vessel for preservation according to the present invention is a vessel for preserving the immersion oil of the present invention mentioned above, it is characterized in that it contains an adsorbent which is always contacted with the immersion oil whenever the immersion oil is taken out. An adsorbent, as mentioned above, silica gel, activated carbon, alumina, clay and the like can be used. It is desired that silica gel or activated carbon is used.

As to a form for containing an adsorbent, it is not particularly limited, if it is a form that adsorbent is not mixed when using an immersion oil. However, it is desirable to constitute such that for example, as shown in FIG. 2, adsorbent 3 is contained in nozzle part 2 of the immersion oil vessel, and the immersion oil 4 is contacted with adsorbent 3 whenever immersion oil 4 is taken out. It is more desirable to constitute such that nozzle part 2 containing adsorbent 3 is detachable to the main part 1 of the preservation vessel, and by changing the nozzle part 2 whenever it is used, immersion oil 4 is always contacted with new adsorbent 3. According to the preservation vessel of the immersion oil of the present invention, for example even if iodine is separated by volatilization or photodegradation of diiodomethane contained in the immersion oil, the separated iodine can be adsorbed with the adsorbent.

Hereafter, the present invention will be explained in detail by embodiments of the invention. The present invention is not limited to the following embodiments.

### Embodiment 1

The immersion oil of the embodiment 1 was manufactured via the following purification process.

Commercially available diiodomethane (Methylene Iodide) (product of Nacalai Tesque. Inc. Diiodemethane GR) was distilled under nitrogen atmosphere and normal pressure (bp. 180-183° C.). Furthermore, 10 g of commercially available sulfur was dissolved in 100 ml of toluene while heating at 80° C., then recrystallized by cooling to room temperature, and obtained solid was filtered and collected. This recrystallized sulfur (2 g) was ground, and, it was suspended in the diiodomethane (10 ml) distilled under normal pressure, and then these were stirred for 17 hours at room temperature, and filtered after stir.

### Embodiment 2

The immersion oil of the embodiment 2 was manufactured via the following purification process.

Commercially available sulfur (4 g) which was ground was suspended in commercially available diiodomethane (20 ml), and these were stirred for 17 hours at room temperature, and filtered after stir. This filtered liquid (10 ml) was refined through a silica gel column chromatography in which silica gel (silica gel 60; product of Nacalai tesque Inc.; ball shape, neutral) (2 g) (an outward volume of 4 ml) was packed.

### Embodiment 3

The immersion oil of the embodiment 3 was manufactured through the following purification process.

Commercially available immersion oil (product of Cargille Co., Series M 1.7800±0.005 No. 0805, refractive index; 1.78) having diiodomethane and sulfur as a main ingredient were refined through a silica gel column chromatography in which silica gel (silica gel 60; product of Nacalai tesque Inc.; ball shape, neutral) (2 g) (an outward volume of 4 ml) was packed.

### Embodiment 4

The immersion oil of the embodiment 4 was manufactured via the following purification process.

Commercially available sulfur (4 g) which was ground was suspended in commercially available diiodomethane (20 ml), and these were stirred for 17 hours at room temperature, and filtered after stir. Activated carbon (product of Nacalai tesque Inc.) (1 g) was suspended in this filtered liquid (10 ml), and it was stirred for 17 hours at room temperature, and filtered after stir.

### Embodiment 5

The immersion oil of the embodiment 5 was manufactured via the following purification process.

Activated carbon (product of Nacalai tesque Inc.) (1 g) was suspended in commercially available immersion oil (product of Cargille Co., Series M 1.7800±0.005 No. 0805, refractive index; 1.78) having diiodomethane and sulfur as main ingredient, and these were stirred for 17 hours at room temperature, and filtered after stir.

### Embodiment 6

The immersion oil of the embodiment 6 was manufactured via the following purification process.

Commercially available diiodomethane was distilled under nitrogen atmosphere (bp. 180-183° C.). Commercially available sulfur (2 g) which was ground was suspended in the diiodomethane (10 ml) which was distilled, and these were stirred for 17 hours at room temperature, and filtered after stir.

### Embodiment 7

The immersion oil of the embodiment 7 was manufactured via the following purification process.

Commercially available sulfur (10 g) was dissolved in 100 ml of toluene while heating at 80° C., then recrystallized by cooling to room temperature, and obtained solid was filtered and collected.

The recrystallized sulfur (2 g) was ground, and it was suspended in the commercially available diiodomethane (10 ml) distilled, and then these were stirred for 17 hours at room temperature, and filtered after stir.

### Embodiment 8

The immersion oil of the embodiment 8 was preserved in the following preservation vessel.

The immersion oil (20 ml) manufactured by the production method of the embodiment 1, leaved as it was for six months at room temperature in a brown bottle placed in the main body of the preserved vessel in which the nonwoven fabric bag containing silica gel (0.4 g; outward volume 8 ml) having basic constitution similar to form shown in FIG. 1 was arranged

### Embodiment 9

The immersion oil of the embodiment 9 was preserved in the following preservation vessel.

The immersion oil (20 ml) manufactured by the production method of the embodiment 1 was put into a brown bottle in a state such that the immersion oil and silica gel were not contacted with, and leaved as it was for 6 months at room temperature. Then, the immersion oil (1 ml) was taken up through the nozzle part 2

### Embodiment 10

The immersion oil of the embodiment 10 was stored in the following preservation vessel.

The immersion oil (20 ml) manufactured by the production method of the embodiment 1, was put into a brown colored bottle in which the nozzle part 2 that contained silica gel (0.5 g; an outward volume 4 ml) as adsorbent 3 and was detachable, was arranged at the main body of the preserved vessel 1. The form and basic composition of the nozzle part were almost the same as shown in FIG. 2. Then, the immersion oil (15 ml) was taken up through the nozzle part 2, and immediately after the nozzle part 2 was replaced by a nozzle part 2 which contained new silica gel the immersion oil (1 ml) was taken up through the nozzle part 2

### Comparative Example 1

The immersion oil in the comparative example 1, is a commercially available immersion oil having refractive index; 1.78 and diiodomethane and sulfur as main ingredients.

### Comparative Example 2

The immersion oil in the comparative example 2 is commercially available immersion oil in which a commercially available sulfur (2 g) which was ground was suspended in commercially available diiodomethane (10 ml), and these were stirred for 17 hours at room temperature, and filtered after stir.

### Comparative Example 3

The immersion oil in the comparative example 3 is an immersion oil manufactured by the production method the embodiment 1 which was put in a brown bottle in which adsorbent such as silica gel etc., is not contained, and leaved as it was for six months at room temperature.

### Comparative Example 4

The immersion oil in the comparative example 4, is an immersion oil, wherein the immersion oil (20 ml) manufactured by the production method of the embodiment 1, was put into a brown bottle (the form and basic composition of the nozzle part were almost the same as shown in FIG. 2.) in which the nozzle part 2 that contained silica gel (0.5 g; an outward volume 4 ml) and was detachable, was arranged and leaved as it was for 6 months at room temperature, and then, after the immersion oil (15 ml) was taken up through the nozzle part 2, the immersion oil (1 ml) was taken up through the nozzle part 2 without changing the nozzle part 2.

With respect to the immersion oil (1 ml) which is immediately after the manufacture in the embodiments 1-7; the immersion oil (1 ml) picked out from the brown bottle in the embodiment 8; the immersion oil (1 ml) picked out from the brown bottle in Embodiments 9 and 10; the immersion oil (1 ml) of the comparative examples 1 and 2, the immersion oil (1 ml) picked out from the brown bottle of the comparative example 3, and the immersion oil (1 ml) picked out from the preservation vessel of the comparative example 4, excitation light of 488 nm was irradiated respectively, and autofluorescence emitted by the irradiation was measured, and then, ratios of the autofluorescence when the autofluorescence of the immersion oil of the comparative example 1 is set to 1 were obtained. The ratios of the autofluorescence are shown in the following table 1.

Here, the fluorescence spectrophotometer F4500 (registered trademark) by Hitachi, Ltd. was used for fluorescence spectrophotometer. Each of 1 ml of the immersion oils of each of embodiments and comparative examples was put into the cell of the fluorescence spectrophotometer for every measurement, and was measured by excitation of 488 nm, and then, and then, ratios were obtained on the basis of the autofluorescence when the autofluorescence of the immersion oil of the comparative example 1 was measured. Here, autofluorescence is included in a cell, and the like of the fluorescence spectrophotometer when the autofluorescence of the immersion oil is measured. However, the measurement was carried out by canceling the autofluorescence as offset.

TABLE 1

| | | Autofluorescence |
|---|---|---|
| | | excited by 488 nm: = |
| | Refractive index | B′/B |
| Embodiment 1 | 1.78 | ≦0.1 |
| Embodiment 2 | 1.78 | ≦0.1 |
| Embodiment 3 | 1.78 | 0.2 |
| Embodiment 4 | 1.78 | 0.4 |
| Embodiment 5 | 1.78 | 0.4 |
| Embodiment 6 | 1.78 | 0.5 |
| Embodiment 7 | 1.78 | 0.5 |
| Embodiment 8 | 1.78 | 0.2 |
| Embodiment 9 | 1.78 | ≦0.1 |
| Embodiment 10 | 1.78 | ≦0.1 |
| comparative | 1.78 | 1 |
| example 1 | | |
| comparative | 1.78 | 1 |
| example 2 | | |
| comparative | 1.78 | 1 |
| example 3 | | |
| comparative | 1.78 | 1 |
| example 4 | | |

As shown in Table 1, unlike the immersion oil of comparative examples, the immersion oil of each of embodiments mentioned above has very small autofluorescence in 488 nm excitation. Accordingly, according to the immersion oil of each of embodiments mentioned above, the weak fluorescence observation of one particle fluorescence etc., observation of which has been difficult or impossible so far can be attained. Moreover, according to the preservation vessel of the immersion oil of the embodiment 8-embodiment 10, in comparison with the preservation vessel of the immersion oil of the comparative examples 3 and 4, degradation of the immersion oil by passage of time can be suppressed, and the autofluorescence in 488 nm excitation can be stopped very small like the immersion oil of the embodiment 1 mentioned above.

The immersion oil, the production method of the immersion oil, the method of preservation of the immersion oil, and the preservation vessel of the immersion oil according to the present invention are useful in fields of biology and medicine, for example, where weak fluorescence observation (or measurement) such as one particle fluorescence observation (or measurement)) is carried out.

What is claimed is:

1. An immersion oil that has refractive index which is 1.70 or more, and low autofluorescence.

2. The immersion oil according to claim 1, wherein iodine compound is main ingredient.

3. The immersion oil according to claim 2, wherein the iodine compound is diiodomethane.

4. The immersion oil according to claim 3, wherein a solid having a high refractive index is dissolved or distributed.

5. The immersion oil according to claim 4, wherein the solid having a high refractive index is sulfur.

6. An immersion oil that has a refractive index which is 1.78 or more, and is low autofluorescence.

7. The immersion oil according to claim 6, wherein iodine compound is main ingredient.

8. The immersion oil according to claim 7, wherein the iodine compound is diiodomethane.

9. The immersion oil according to claim 8, wherein a solid having a high refractive index is dissolved or distributed.

10. The immersion oil according to claim 9, wherein the solid having a high refractive index is sulfur.

11. A production method of the immersion oil according to the claim 1, wherein the production method has a purification process.

12. The production method of the immersion oil according to claim 11, wherein the purification process is a process for removing impurities by adsorbent.

13. The production method of the immersion oil according to claim 11, wherein the purification process is a process for removing impurities by distillation.

14. The production method of the immersion oil according to claim 11, wherein the purification process is a process for removing impurities by recrystallization.

15. A production method of the immersion oil according to claim 6, wherein the production method has a purification process.

16. The production method of the immersion oil according to claim 15, wherein the purification process is a process for removing impurities by adsorbent.

17. The production method of the immersion oil according to claim 15, wherein the purification process is a process for removing impurities by distillation.

18. The production method of the immersion oil according to claim 15, wherein the purification process is a process for removing impurities by recrystallization.

19. A method of preservation of the immersion oil according to claim 1, wherein an adsorbent is always contacted with the immersion oil.

20. A method of preservation of the immersion oil manufactured by the production method according to claim 11, wherein an adsorbent is always contacted with the immersion oil.

21. A method of preservation of the immersion oil according to claim 1, wherein the immersion oil is contacted with an adsorbent whenever it is taken out from the immersion oil to the exterior of the preservation vessel.

22. A method of preservation of the immersion oil manufactured by the production method according to claim 11 wherein the immersion oil is contacted with an adsorbent whenever it is taken out from the immersion oil to the exterior of the preservation vessel.

23. A preservation vessel of the immersion oil according to claim 1, wherein the preservation vessel contains an adsorbent which is always contacted with the immersion oil.

24. A preservation vessel of the immersion oil manufactured by the production method according to claim 11, wherein the preservation vessel contains an adsorbent which is always contacted with the immersion oil.

25. A preservation vessel of the immersion oil according to claim 1, wherein the preservation vessel contains an adsorbent which is contacted with the immersion oil whenever it is taken out from the immersion oil in the preservation vessel.

26. A preservation vessel of the immersion oil manufactured by the production method according to claim 11, wherein the preservation vessel contains an adsorbent which is contacted with the immersion oil whenever it is taken out from the immersion oil in the preservation vessel.

27. The preservation vessel of the immersion oil according to claim 26, wherein a portion which contains an adsorbent contacted with the immersion oil whenever it is taken out can be attached and detached to the main body of the preservation vessel.

28. The preservation vessel of the immersion oil according to claim 25, wherein a portion which contains an adsorbent contacted with the immersion oil whenever it is taken out can be attached and detached to the main body of the preservation vessel.
